(19) United States
(12) Patent Application Publication
(10) Pub. No.: US 2004/0115277 A1
(43) Pub. Date: Jun. 17, 2004

(54) Microparticles with an improved release profile and method for the production thereof

(76) Inventors: Thomas Kissel, Staufen im Breisgau (DE); Ruland Fridrich, Blaubeuren (DE); Peter Schneider, Dornburg-Thalheim (DE)

Correspondence Address: Christopher E Aniedobe, Reed Smith, East Tower Suite 1100, 1301 K Street, Washington, DC, 20005-3373, US

(21) Appl. No.: 10/450,407

(22) PCT Filed: Dec. 11, 2001
(86) PCT No.: PCT/EP01/14515

(30) Foreign Application Priority Data
Dec. 13, 2000 (DE) 100 61 944.4
Apr. 11, 2001 (DE) 101 18 160.4

(51) Int. Cl. A61K 9/14

(52) U.S. Cl. 424/489

(57) ABSTRACT

The present invention relates to microparticles for the delayed release of a physiologically active ingredient, said particles containing at least one active ingredient and a polymer matrix. The microparticles of the present invention possess particularly advantageous release characteristics. The present invention also relates to a method for manufacturing microparticles of the aforementioned kind.

The present invention relates to microparticles used in the delayed release of a physiologically active ingredient and which contain at least one active ingredient and a polymer matrix. The microparticles according to the present invention possess particularly advantageous release characteristics. The present invention also relates to a method for manufacturing microparticles of the aforementioned kind.

When administering drugs over a longer period of time, it is frequently desirable to maintain a maximally constant plasma level of the active ingredient. This is especially difficult to achieve if the corresponding active ingredient quickly disintegrates or precipitates out in the body. In order to avoid repeated applications at short intervals, various depot drugs have been proposed, which aim at releasing a maximally constant amount of active ingredient over a longer period of time. Depo drugs of this type often take the form of microparticles that are administered parenterally, e.g. implanted or injected subcutaneously. Generally, such drugs comprise a polymer matrix within which the active ingredient is dispersed (“microspheres”), or they comprise a core containing the active ingredient, which is surrounded by a polymer-laden coating (microcapsules).

Various methods are known in the prior art for manufacturing microparticles.

In the so-called W/O/W method an initial aqueous phase containing the active ingredient is dispersed in an organic polymer solution (O), after which the resultant W1/O-emulsion is dispersed in a second aqueous phase (so-called outer phase; W2). The polymer is coacervated through removal of the organic solvent, thereby forming microparticles. Particle size is influenced by the respective dispersion process used. Finally, the formation of a microparticle is also a function of the evaporation potential of the solvent. For this reason, the W/O/W double emulsion method is also termed “solvent evaporation/extraction method/technique”. Once the microparticles are cured and the solvent removed, microparticles are obtained, which contain the active substance. Frequently, microparticles of this type contain viscosity-enhancing substances such as, for example, gelatin.

S/O/W methods are also known from the prior art, in which the active substance is present in a solid (S), rather than in an aqueous solution. The solid is then directly dispersed in the organic phase (O). The subsequent steps are identical to those of the W/O/W method.

Finally, there are so-called S/O/O methods in which the outer phase, instead of being an aqueous phase, is a non-aqueous phase containing a protective colloid or an emulsifier.

It is desirable to keep the amount of microparticles to be administered to the patient as minimal as possible. For example, the volume of microparticles to be administered should be as minimal as possible, amongst others, in order to lessen the pain associated with the injection. Hence, the content of active substance within the microparticles should be as high as possible. Ingredient load is an important characteristic of microparticles. A differentiation is made between actual and theoretical load degree. Terms used as synonyms for actual load degree are effective load degree or effective ingredient content. Theoretical load degree is defined as follows:

$$Theoretical load degree in \% = \frac{Mass of active ingredient \times 100}{Mass ( active ingredient + polymer + load material )}$$

Involved here is the mass of the components used in the manufacturing process. Effective active ingredient content is defined as follows:

$$Effective ingredient content in \% = \frac{Mass of active ingredient in mg \times 100}{Weight of microparticles in mg}$$

The ratio of effective ingredient content to theoretical load degree is referred to as encapsulation yield. Encapsulation yield is an important process parameter and a measure of the method's effectiveness:

$$Encapsulation yield in \% = \frac{Effective ingredient content \times 100}{Theoretical load degree}$$

Another important criterion is the release profile of the microparticle. The release of the active ingredient can be subdivided into roughly three temporal phases. In an initial “burst” phase, substantial quantities of the active ingredient contained in the microparticles are normally released in a relatively short period of time. This involves, in part, active ingredient disposed at or near the surface of the particles. The amount of active ingredient released during the “burst”-phase should be as minimal as possible. In the ensuing “lag”-phase, the release of active ingredient in prior art preparations has been negligibly small, especially when employing PLGA-polymers as a matrix former. It would be desirable during the “lag”-phase to have a maximally constant delivery of active ingredient throughout the release period. In the final bio-erosion phase, the particles are hydrolyzed and release increased amounts of active ingredient as a result of significant loss in mass and molecular weight. Ideally, the entire amount of active ingredient would be released as early as during the “lag”-phase.

Kishida et al. (1990) J. Controlled Release 13, 83-89 investigates the effect of load degree, active ingredient lipophilia and rate of solvent removal on the lipophilic substance Sudan III, versus the polar etoposide. It was found that when using polyvinyl alcohol as a stabilizer, the removal of solvent using different vacuum settings during the curing phase had no effect on release.

For a W/O/W-procedure using PLGA for encapsulating gp120, Cleland et al. (1997) J. Controlled Release 47, 135-150, investigates the effect of kinematic viscosity of the polymer in the primary emulsion and use of excess dichloromethane in the outer phase on ingredient load and release during the “burst”-phase.

An object of the present invention is to prepare microparticles that have an advantageous release profile.

It was found unexpectedly that microparticles exhibiting a higher total release could be obtained if the outer phase to which the primary emulsion is added was pre-cooled. In the present application, the total exploitable release is that percentage of the total amount of active ingredient contained in the microparticles that is released within 900 hours from the onset of release. It was also found that the amount of active ingredient released during the “burst”-phase may be significantly reduced through accelerated removal of the organic solvent. This occurs either by dispersing the primary emulsion in the outer phase and subjecting the emulsion or dispersion product to low pressure, or by conducting an inert gas through the emulsion or dispersion product, resulting in the accelerated removal of the organic solvent.

The present invention also relates to a method for manufacturing microparticles for the delayed release of an active ingredient, characterized in that

a) a composition containing the active ingredient is added to an organic polymer solution and dispersed therein,

b) the emulsion or dispersion produced in a) is added to an outer phase and dispersed therein, wherein the temperature of the outer phase at the time of admixture is between 0° C. and 20° C., and

c) the organic solvent is removed by subjecting the dispersion or emulsion product of b) to a pressure of less than 1,000 mbar, or by conducting an inert gas into the dispersion or emulsion product of b).

Any physiologically active substance may be used as an active ingredient in the microparticles. It is preferable if these are water-soluble substances. Examples of active ingredients that can be used are immunizing agents, antitumor drugs, antipyretics, analgesics, anti-inflammatory substances, active substances effecting blood coagulation, such as Heparin, Antitussiva, Sedativa, muscle relaxants, antiulceratives, antiallergics, vasodialators, antidiabetics, antituberculosis drugs, hormone preparations, contraceptives, bone resorption inhibitors, angiogenesis inhibitors, etc. Normally, active ingredients in the form of peptides or proteins are used. Examples of potential peptide- or protein-based active ingredients are salmon-calcitonin (sCT), lysozyme, cytochrome C, erythropoietin (EPO), luteinizing hormone releasing hormone (LHRH), buserelin, goserelin, triptorelin, leuprorelin, vasopressin, gonadorelin, felypressin, carbetocin, bovine serum albumin (BSA), oxytocin, tetanus toxoid, bromocriptin, growth hormone releasing hormone (GHRH), somatostatin, insulin, tumor necrosis factor (TNF), colony stimulating factor (CSF), epidermal growth factor, (EGF), nerve growth factor (NGF), bradykinin, urokinase, asparaginase, neurotensin, substance P, kallikrein, gastric inhibitory polypeptide (GIP), growth hormone releasing factor (GRF), prolactin, adrenocorticotropes hormone (ACTH), thyrotropin releasing hormone (TRH), thyroid stimulating hormone (TSH), melanocyte stimulating hormone (MSH), parathormone (LH), gastrin, glucagon, enkephalin, bone morphogenetic protein (BMP), α-, β-, γ-interferon, angiotensin, thymopoetin and thymic humor factor (THF).

Active ingredients in the form of peptides or proteins may derive from a natural source or they may be recombinantly produced and isolated. Recombinantly produced ingredients may differ from their counterpart native ingredients, for example, in the type and extent of posttranslational modifications, as well as in the primary sequence. Such modified active ingredients may also possess other properties, such as altered pharmacological efficacy, altered precipitation behavior, etc. All such “variants” of naturally occurring active ingredients fall within the scope of the present invention. Other potential active ingredients include heparin and nucleic acids such as DNA and RNA molecules. DNA molecules may be present either in linear or circular form. Plasmids or vectors, in particular expression vectors, may also be included. An example thereof is the expression vector pcDNA3 described in international patent publication WO/98/51321.

Finally, viral vectors of the type used in gene therapy are also encompassed by the present invention. In addition, complexes composed of chitosan, sodium-alginate or other cationic polymers such as polyethylenimine or poly(lysine) or other cationic amino acids may be used. The nucleic acids used may be single or double-stranded. Single-stranded DNA may be used, e.g. in the form of antisense-oligonucleotides. Further, “naked” nucleic acid fragments may be used; in which case the nucleic acids are not bound with other materials.

The concentration of active ingredient is dependent among other things on the respective ingredient and type of treatment for which it is being employed. As a rule, peptide/protein ingredients are used in a concentration of 0.01 to 30%, preferably from 0.5 to 15%, primarily from 1.0 to 7.5%, relative to the polymer mass used.

The function of the organic phase, non-miscible with water, is to dissolve the biologically degradable polymer. In this process the polymer is dissolved in a suitable organic solvent in which the active ingredient is indissoluble. Examples of organic solvents of this type are ethyl acetate, acetone, dimethyl sulfoxide, toluol, chloroform, ethanol, methanol, etc. Dichloromethane is especially preferred. The concentration of polymer in the organic phase is normally greater than 5% (w/v), preferably 5 to 50%, most preferably 15 to 40%.

Any biodegradable and biocompatible polymer may be used to form the polymer matrix of the microparticles. The former may be naturally occurring or of synthetic origin. Examples of naturally occurring polymers are albumin, gelatine and carragen. Examples of synthetic polymers which may be used in the method according to the present invention are polymers derived from fatty acids (e.g. polylactic acid, polyglycolic acid, polycitric acid, polymalic acid, polylactic acid caprolacton, etc.), poly-α-cyanoacryl acetic acid, poly-β-hydroxy butric acid, polyalkylene oxalate (e.g. polytrimethylene oxalate, polytetramethylene oxalate, etc.), polyorthoester, polyorthocarbonate and other polycarbonates (e.g. polyethylene carbonate, polyethylene propylene carbonate, etc.), polyamino acids (e.g. poly-γ-methyl-L-glutamine acid, poly-L-alanine, poly-γ-methyl-L-glutamic acid, etc.), and hyaluronic acid esters, etc. Other bio-compatible copolymers are polystyrol, polymethacrylic acid, copolymers made of acrylic acid and methacryclic acid, polyamino acids, dextranstearate, ethylcellulose, acetylcellulose, nitrocellulose, maleic anhydride-copolymers, ethylene-vinylacetate-copolymers, such as polyvinylacetate, polyacrylamide, etc. The aforementioned copolymers may be used alone or in combination with one another. They may be used in the form of copolymers or as a mixture of two or more of the polymers. It is also feasible to utilize the salts derived therefrom. Among the polymers cited, lactic acid/glycolic acid-copolymers (PLGA) are preferred. Preferable are PLGA-polymers with a lactic acid to glycolic composition ratio ranging from 0:100 to 100:0 and a molecular weight of 2,000 to 2,000,000 Da. Especially preferred are PLGA-polymers having a molecular weight of 2,000 to 200,000 Da and a lactic acid/glycolic acid ratio ranging from 25:75 to 75:25 or 50:50. L-PLA or D,L-PLA or mixtures or copolymers thereof may also be used.

The composition containing the ingredient may be an aqueous solution, for example when employing the W/O/W-method. In such case, the active ingredient is normally dissolved in water or a buffer solution and dispersed directly in the organic polymer solution. The resulting W1/O—or primary emulsion is then injected in the outer phase (W2) which optionally contains a protective colloid, and dispersed using conventional agents. The product of this step is the double emulsion or W1/O/W2-emulsion. Following a curing phase the resultant microparticles are separated from the outer aqueous phase and may be subsequently lyophilized. Microcapsules are obtained by the W/O/W-method from large W1-volumes and with a low viscosity polymer solution. For example, a volume ratio W1:O:W2 of 1:10:1000 would result in the formation of “microspheres”, a volume ratio of 9:10:1000 would result in the formation of microcapsules.

However, the composition containing the active ingredient may also occur in solid form. In this case, the active ingredient is dispersed in solid form directly in the polymer solution. The further manufacturing steps are identical to those of the W/O/W-method. Utilizing additional method steps, it is possible to apply either the S/O/W- or S/O/O-method.

In certain embodiments of the method according to the present invention the outer phase is an aqueous solution (W2). Such an aqueous phase may contain an emulsifier or a protective colloid. Examples of protective colloids are polyvinyl alcohol, polyvinylpyrrolidone, polyethylene glycol, etc. Polyvinyl alcohol is preferred. By way of example, several of the polyvinyl alcohols of available from Clariant may be used, such as Mowiol® 18-88, Mowiol® 4-88, or Mowiol® 20-98. The protective colloids are normally used in a concentration of 0.01% to 10%, preferably 0.01% to 5%. The molecular weight of the protective colloids may range from between 2,000 and 1,000,000 Da, preferably between 2,000 and 200,000 Da. The W1/O-primary emulsion and outer phase should have a volume ratio relative to one another ranging from 1:5 to 1:1,000.

As an alternative, it is also feasible to employ a so-called “oily” phase that is non-miscible with the primary emulsion (“W/O/O-, respective S/O/O-method”). For example, it is possible to use silicone oil or paraffin oil which contain an emulsifier and/or a protective colloid. Unlike the use of an aqueous outer solution, an “oily” outer phase requires the presence of an emulsifier or a protective colloid. Examples of emulsifiers in the outer oily phase are Span, Tween or Brij, preferably in a concentration of from 0.01 to 10 percent by weight.

According to the present invention the temperature of outer phase ranges between 0 to 20° C. when the primary emulsion is added to and dispersed in said outer phase. Preferably, said temperature ranges between 0° C. to 10° C., more preferably between 3° C. to 7° C., most preferably around 5° C. It is also preferred if the resultant emulsion or dispersion is next subsequently regulated in the aformentioned temperature ranges, e.g. in a laboratory reactor. It is most preferable for the temperature according to the present invention to be maintained subsequent to dispersion of the primary emulsion in the outer phase until such time as the microparticles are fully cured.

According to the present invention, removal of the organic solvent is also accelerated. This can be achieved by subjecting the emulsion or dispersion produced by dispersion of the primary emulsion in the outer phase to low pressure, that is, to a pressure lower than atmospheric pressure. In accordance with the present invention, the emulsion or dispersion may be subjected to a pressure of less than 1,000 mbar, preferably a pressure of 500 mbar or less, most preferably a pressure of 50 to 150 mbar. This vacuum accelerates the removal of the organic solvent. Said vacuum may be advantageously applied during the curing of the microparticles, when using a laboratory reactor for manufacturing the microparticles. Instead of applying a low pressure, it is also possible to accelerate the removal of the organic solvent by conducting an inert gas into the emulsion or dispersion. Inert gases e.g. in the form of rare gases may be used, though nitrogen is preferred. Injection of nitrogen accelerates the removal of the volatile organic solvent.

In an especially preferred embodiment of the present invention, the microparticles are cured at low temperature, that is, in a temperature range of between 0° C. and 10° C., preferably around 5° C. and under reduced pressure, that is, at a pressure of 500 mbar or less. It is especially preferable to apply a vacuum in this instance, that is, a pressure of between about 50 and about 100 mbar.

It was also found that the presence of chitosan in the microparticles allows for higher load degrees of active ingredient than is the case with microparticles according to the prior art. It is thus feasible to use chitosan in the manufacture of microparticles according to the present invention. Chitosan is a polymer obtained by deacetylizing chitin, a polysaccharide occurring in insects and crustacean. Normally, it is a linear-chained polysaccharide constructed from 2-amino-2-desoxy-β-D-glucopyranose (GlcN), in which the monomers are β-(1,4)-linked (100% deacetylization). In the case of incomplete deacetylization, chitosan preparations are produced that still exhibit different quantities of 2-acetamido-2-desoxy-β-D-glucopyranose (GlcNAc) in the polysaccharide chain.

According to the present invention, the chitosan may exhibit varying degrees of deacetylization. Virtually 100% deacetylized Chitosan contains essentially just GlcN and no longer any GlcNAc. Preferably, the chitosan according to the present invention is deacetylized to a degree of from 25 to 100%, most preferably from 50 to 100%.

The weight ratio of physiologically active ingredient to chitosan is preferably 1:0.01 to 1:25, more preferably 1:0.01 to 1:10, most preferably 1:1. The ratio is indicated in wt/wt.

Normally, chitosan with a molecular weight of 10,000 to 2,000,000 Da is used, preferably of 40,000 to 400,000 Da. Chitosan is usually dissolved in a 0.001% to 70% acetic acid solution, preferably in a 0.01% to 10% acetic acid solution (m/m). According to the present invention the particles may be manufactured by the W/O/W-, S/O/W- or S/O/O-methods. The active ingredient may be dissolved with chitosan in acetic acid, or first dissolved in water, then dispersed with the dissolved chitosan. The chitosan-active ingredient gel is then directly dispersed in the organic polymer solution (W/O/W). It is also feasible to spray-dry the chitosan-active ingredient-solution, then directly disperse the solid powder in the organic polymer solution (S/O/W; S/O/O).

The concentration of chitosan in the inner phase under the W/O/W method is generally 0.01% to 50%, relative to polymer mass, but preferably 0.01% to 25% chitosan, relative to polymer mass. The weight ratio of physiologically active ingredient to chitosan should range from 1:0.01 to 1:25, preferably from 1:0.1 to 1 :10, most preferably 1:1. Under the S/O/W-method a concentration of chitosan ingredient complex ranging from 0.01% to 50%, preferably 0.1% to 25% relative to polyer mass should be used.

The present invention also relates to microparticles that may be manufactured by the method according to the present invention. Microparticles of this type have release profiles that exhibit advantages properties. Thus, for example, the amount of active ingredient released during the “burst”-phase is very small. Also, a large portion of the active ingredient contained in the microparticle is released during the “lag”-phase. Thus, there is overall a very high release of active ingredient. Accordingly, the present invention concerns microparticles containing a polymer matrix and at least one physiologically active ingredient, characterized in that according to the in vitro release profile of said microparticles

a) within 24 hours of the onset of release less than 25% of the total amount of active ingredient is released; and

b) within 900 hours of the onset of release, at least 80% of the total amount of active ingredient has been released.

Data on the release of active ingredient in this application pertain to the release determined in vitro in a release apparatus in accordance with the method described in Example 5. It is known that the release of active ingredient under the aforementioned in vitro-method closely approximates the release in vivo.

Microparticles with this kind of advantageous release profile are currently unknown in the prior art. Prior art microparticles exhibit a relatively high release during the “burst”-phase and/or very low release during the “lag”-phase, resulting in a low overall release. The risk created by this is that not until the following bio-erosion phase is a large quantity of active ingredient once again released.

The microparticles according to the present invention release within 24 hours of the onset of release less than 25% of the total amount of active ingredient, preferably less than 20%, most preferably less than 15%.

Likewise, another property of said microparticles is that within 900 hours of the onset of release at least 80% of the total amount of active ingredient contained therein is released, preferably at least 85%, most preferably at least 90%.

The microparticles according to the present invention exhibit within a period of between 48 and 900 hours after the onset of release, preferably within a period of 24 to 900 hours after the onset of release, a release that is kinetically substantially on the order of zero. This means that over a period of more than 30 days, each day a substantially constant amount of active ingredient is released. Preferably, 1.5% to 2.5% of the total amount of active ingredient is released in the period of between 48 and 900 hours after onset of release, preferably, 2% to 2.5%.

Generally, the microparticles according to the present invention have a diameter of between 1 and 500 μm, preferably between 1 and 200 μm, still more preferably between 1 and less than 150 μm, most preferably between 1 and 100 μm. They may be spherical or they may vary in shape. For particles that are not spherical in shape, diameter is defined as the largest spatial extension of a particle. The polymer matrix may be in the form of a shell that surrounds the core, or as a “framework” that permeates the entire particle. Accordingly, the microparticles according to the present invention comprise both particles that have a core containing the active ingredient and are surrounded by a polymer coating (microcapsules) as well as particles that have a polymer matrix within which the active ingredient is dispersed (“microspheres”).

In a separate embodiment of the invention the microparticles may also contain chitosan. The properties of chitosan and the concentrations according to the present invention are indicated above. Particles of this type exhibit an overall greater effective load degree of active ingredient.

Another aspect of the present invention is a pharmaceutical that includes the microparticle according to the present invention, optionally including pharmaceutically acceptable excipients.

The present invention makes available for the first time microparticles that combine low release of active ingredient during the “burst”-phase with a high overall release. Moreover, in microparticles according to the present invention the release profile of the active ingredient during the “lag”-phase is substantially linear. The microparticles according to the present invention make possible the release of active ingredient over a period of weeks and even months. Thus, they are particularly suited to subcutaneous/intramuscular application.

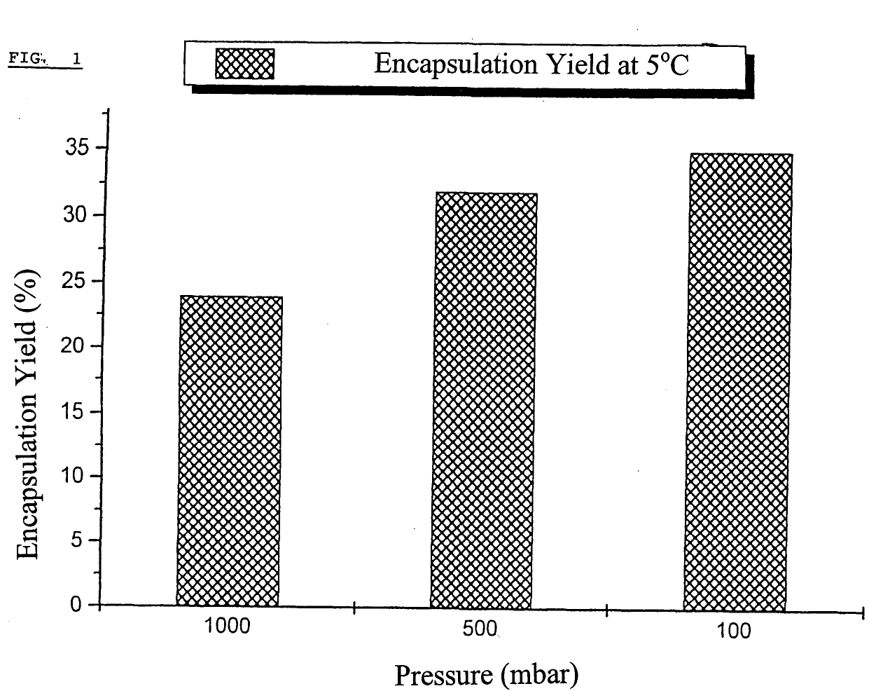

FIG. 1 shows the relationship between encapsulation yield (EY) and pressure applied during the curing of the microparticles in a laboratory reactor at a constant 5° C. Encapsulation yield increases with decreased pressure.

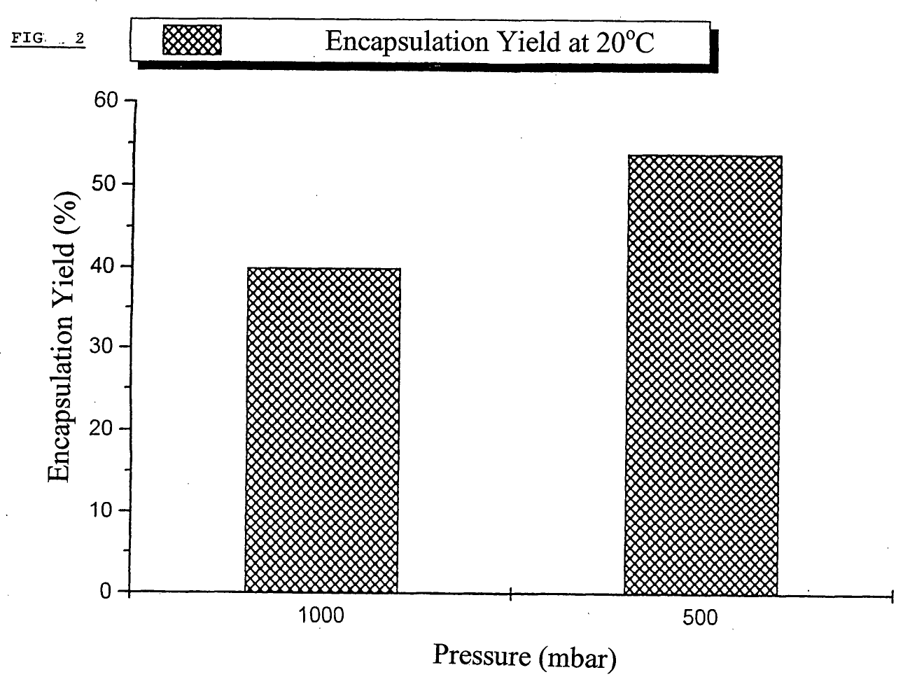

FIG. 2 shows the relationship between encapsulation yield (EY) and pressure applied during curing of the microparticles in a laboratory reactor at a constant 20° C. In contrast to FIG. 1, only two pressures are tested here, namely atmospheric pressure and 500 mbar. Even at 20° C. it is apparent that low pressure during curing produces higher encapsulation yields.

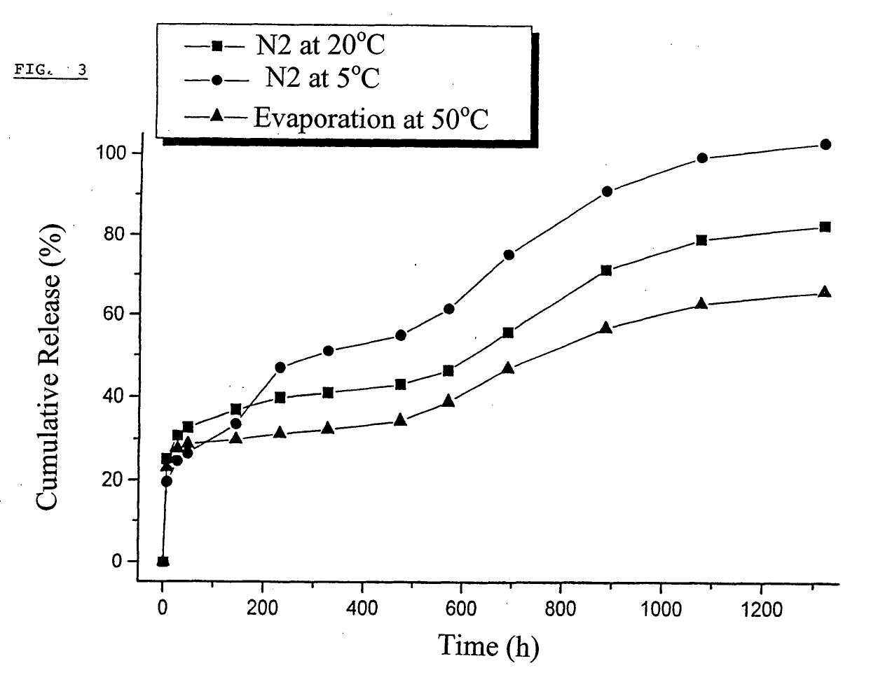

FIG. 3 shows the relationship between the in vitro-release of lysozyme with concomitant injection of nitrogen (N₂) during curing of the microparticles in a laboratory reactor at different temperatures (5° C. and 20° C.). Also shown is the in vitro-release profile of microparticles, in which the solvent was evaporated during the curing phase at 50° C. Here, lower overall release in conjunction with higher temperatures is apparent. Moreover, lowering the temperature from 20° C. to 5° C. results in a 6% lower initial release and an increase in overall release of 99.7% as opposed to 79.3% at 20° C. after 1,074 hours of release. Further, the curve “N₂” at 5° C. evidences a lower release of active ingredient during the “burst”-phase.

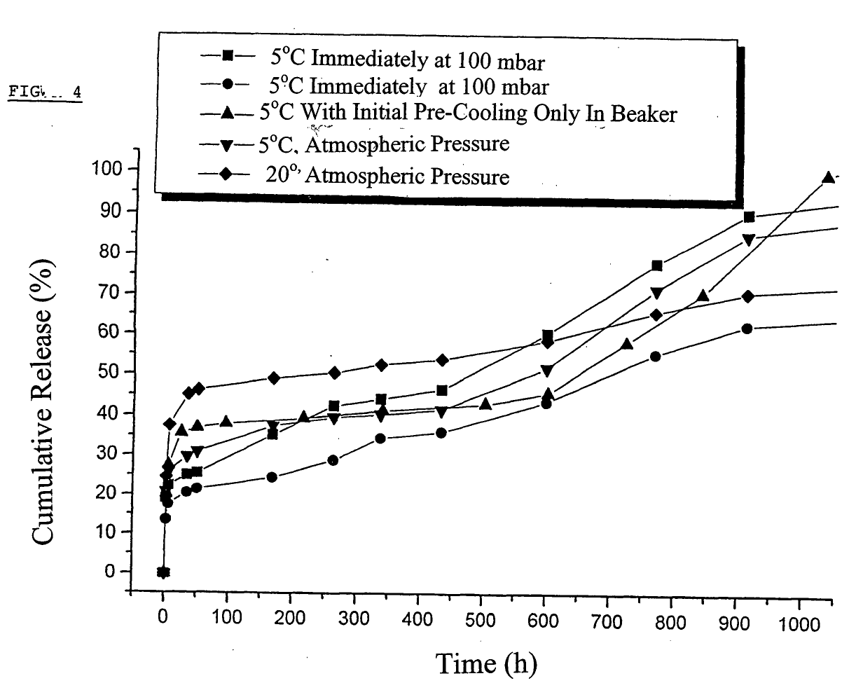

FIG. 4 displays the results of Example 9. The application of low pressure at low temperatures results in a low “burst” of 22.4% at 5° C. after 5 h and in 100 mbar vacuum, and to a higher overall release of 90.5%. At 20° C. and a pressure of 100 mbar the overall release is only 62.8% after 912 hours.

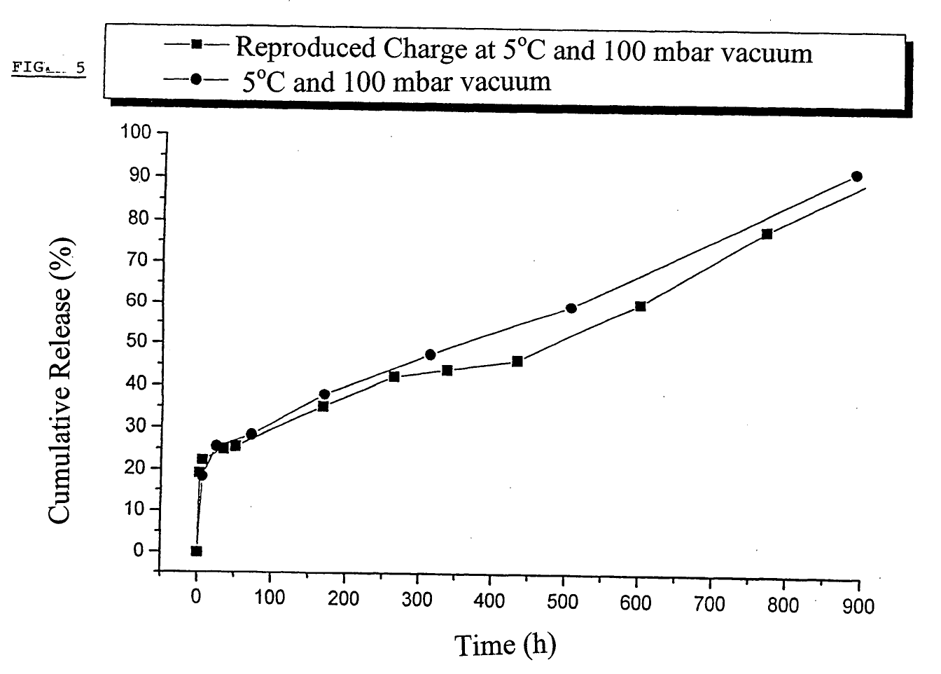

FIG. 5 shows the release profile of two charges prepared independently of one another at 100 mbar and at 5° C. during curing of the microparticles in a laboratory reactor. Thus, it is possible, duplicating the method of the present invention, to manufacture microparticles that have substantially the same release profile. As is apparent from these series of data, the microparticles exhibit a largely linear release.

The following examples elucidate the present invention in greater detail.

### EXAMPLE 1

### Manufacture of Microparticles by the W/O/W-Method

Microparticles with Lysozyme

To manufacture microparticles containing peptides from PLA or PLGA, the following “solvent/evaporation/extraction” method was used: a standard measure of 2.00 g of PLGA-polymer (RG 503 H from Boehringer Ingelheim) in a 20 ml Omnifix syringe with Luer lock and suitable combination closing stopper was fully dissolved in 5.7 ml dichloromethane (DCM) (DCM density=1.32 g/ml [Merck Index]) (35% m/v). 100.00 mg of lysozyme were gently stirred by a magnetic stirrer and dissolved to clarity in a 4 ml HPLC-vial in distilled water or buffer. Next, 1000 μl of the peptide solution are injected into the polymer solution and dispersed using a SN-10 G Ultraturrax-mixer for 60 minutes at 13,500 revolutions per minute (rpm). The primary emulsion (W1/O) is then injected from the Omnifix syringe into 500 ml of a 0.1% polyvinyl alcohol solution pre-cooled to 5° C. (Mowiol 18-88: Mw=130 kDa, 88% degree of hydrolysis) and simultaneously dispersed using the SN-18 G Ultraturrax-mixer for 60 seconds at 13,500 rpm, thereby producing a W1/O/W2-double emulsion. The latter is then cured using an IKA-series stirrer and 2-blade centrifugal stirrers at 240 rpm for 3 hours at room temperature (RT) in open 600 ml beakers under atmospheric pressure.

The entire double emulsion containing the cured microparticles is then placed in centrifuge tubes and centrifuged in the Heraeus Megafuge 1.0 at 3,000 rpm for a period of 3 minutes and the W2-phase residue is then separated off. Subsequently the microparticles are passed over a 500 ml Nutsche filter (borosilicate 3.3; pore density 4) and washed at least 3× in distilled water. The resultant microparticles obtained from the frit are repeatedly suspended in a small amount of distilled water and washed to remove PVA-residues.

The microparticles obtained are collected, then placed in previously tared vessels and lyophilized. The microparticles are then placed in a Delta 1 A apparatus set to operating conditions and subjected to a main drying for at least 120 h at −60° C. and at a 0.01 mbar vacuum. They are then dried a second time for 24 h at 10° C. and in 0.01 mbar vacuum to remove any residual solvent and water. The microparticles are then weighed in the vessels and the yield is calculated.

### EXAMPLE 2

### Manufacture of Microparticles by the S/O/W-Method

Manufacturing takes place under the same conditions used in the W/O/W method with one difference in the first manufacturing step, in which a specific quantity of peptide or protein is not dissolved, but rather is added in lyophilized or spray-dried form directly to the dissolved polymer (35% m/m) in DCM and dispersed for a period of 30 seconds at 13,500 rpm using the SN-10 G Ultraturrax-mixer. The resultant S/O- or primary suspension is then dispersed in the outer phase to produce an S/O/W-emulsion. All further manufacturing steps are performed under conditions analogous to those in the W/O/W-method.

### EXAMPLE 3

### Manufacture of Microparticles using a Laboratory Reactor

An IKA-laboratory reactor LA-R 1000 was used as a process apparatus for manufacturing W/O/W- or S/O/W-microparticles under controlled conditions. The conditions under the W/O/W- or S/O/W-methods were duplicated here (see Example 1 and 2). As part of the process, the primary emulsion is produced in an Omnifix syringe, then injected through one of the openings in the reactor cover into a 0.1 % PVA-solution (500 ml) which was previously placed in the IKA-laboratory reactor and preset to a specific temperature, at the same time being dispersed for a period of 60 seconds using the Ultraturrax T25 and the SN 18 G mixer at 13,500 rpm. Once dispersion is completed, the Ultraturrax is removed from the IKA-reactor and the reactor vessel sealed. At this point a specific pressure may be applied. In the following examples, primarily 500 mbar and 100 mbar were applied, in addition to atmospheric pressure. Next, the microparticles are cured under constant stirring using an anchor stirrer at 40 rpm for 3 h and at a constant temperature. Various temperature settings may be used. Primarily temperatures of 20° C. and 5° C. were used. Separation and lyophilization of the microparticles were carried out in the manner previously described under the W/O/W- and S/O/W-methods.

The apparatus comprises a reactor vessel 1 l in size and may be temperature regulated within the range of −30° C. to 180° C. via a double jacket vessel bottom. The temperature is regulated by means of a circulation thermometer. A vacuum is applied using a Jahnke & Kunkel MZ 2 C vacuum pump. Further, the temperature of the reactor contents, cooling fluid, vacuum, stir rate and rotational rate of the Ultralturrax are measured by sensors (PT 100 for temperature) and transmitted to the software. The process apparatus is controlled using the Software Labworldsoft Version 2.6.

### EXAMPLE 4

### Method for Determining the Ingredient Load of the Microparticles

The ingredient load of the microparticles is determined in accordance with the modified method of Sah et al. (A new strategy to determine the actual Protein Content of Poly(lactide-co-glycolide) Microspheres; Journal of Pharmac. Sciences; 1997; 86; (11); pp. 1315-1318). The microparticles are dissolved in a solution of DMSO/0, 5% SDS/0.1 N NaOH, from which solution a BCA-assay (Lowry et al. “Protein measurement with the Folin Phenol Reagent”; J. Biol. Chem.; 193 pp. 265-275; 1951) is then performed. From this the effective load degree of the microparticles is determined.

### EXAMPLE 5

### Determination of in Vitro-release

The cumulative release of lysozyme as a % of the total amount of lysozyme contained in the microparticles was investigated in the following way:

To determine the release of active ingredient from the microparticles 20 mg increments of the microparticles were weighed (three-fold preparation per charge). The microparticles were then placed in Pyrex test tubes fitted with a Schott-stopper GL18-thread and a Teflon seal. To each microparticle increment 5 ml Mc.Ilvaine-Whiting release buffer (composition, see below) was added, after which the samples were placed in the release apparatus (6 rpm; 37° C.). The release apparatus consists of a universal holding plate made of polypropylene for holding Eppendorf vessels or Pyrex test tubes. The plate can be set in a rotating motion in a temperature controlled housing, so that the vessels rotate about their transverse axes. The rate of rotation may be continuously adjusted from 6-60 rpm. The entire inner space is temperature regulated by warm air circulation. The first sample was removed after two hours, the second after approximately six hours, the third after approximately 24 hours, the fourth after 48 hours and the remaining samples after a period of three days, respectively. The Pyrex test tubes were centrifuged at 3000 rpm (4700 g) for 3 minutes in a Heraeus, Hanau, Megafuge 1.0 centrifuge, after which as much of the remaining buffer as possible was removed with the aid of a Pasteur pipette. Subsequently, 5 ml buffer were again added to the test tubes and the samples were again placed in the release apparatus. The buffer was stored in the dark and refrigerated at 4° C.

Composition of the Mc.Ilvaine-Whiting release buffer:

0.0094 M citric acid

0.1812 M disodium hydrogen phosphate

0.01% (w/v) Tween for the molecular biology)

0.025% (w/v) sodium azide

pH 7.4

in distilled water.

The peptide solution that was pipetted out of the Eppendorf vessels or Pyrex test tubes was transferred to 4 ml HPLC-vials with pierceable Teflon seals and a turn stopper, and either subjected directly to HPLC analysis or stored at −30° C. Prior to HPLC analysis the samples were thawed at room temperature for two hours and shaken several times by hand in the process, making sure that the solution was completely clear after thawing. The HPLC analysis was performed on a Waters HPLC with a W600 pump, 717 autosampler, Satin 474 UV detector and Millenium 3.15 software. The settings for lysozyme were as follows:

Flow rate 1 ml/min

Buffer A=0.1% TFA (trifluoro acetate) in water,

Buffer B=0.1% TFA in Acetonitrile

Gradient: 80% A, 20% B in 10 minutes at 60% A, 40% B; up to 12 minutes at 80% A, 20% B

Excitation wave length=280 nm,

Emission wave length=340 nm at gain=100,

256 attention and STD

Column furnace tempering 40° C.

Column: TSK Gel RP 18, NP; 5 μm; 35 mm×4.6 mm

Prior to analysis the fluid medium was degassed using helium or ultrasound and degassed during analysis using a degaser.

For each sample set, standard series of 0.05 to 4 μg lysozyme/ml of release buffer at 100 μl injection volumes and 10 to 100 μg lysozyme/ml of release buffer at 10 μl injection volume were analyzed as a standard.

The method described above for determining in vitro-release is concerned with lysozyme as the active ingredient and in its present form is not applicable to leuprorelin. For determining other active ingredients such as, for example, leuprorelin, some of the parameters require modification, such as, for example, column used, buffer medium and applied wavelengths. Such modifications however are obvious to one skilled in the art.

### EXAMPLE 6

Here, the effect of reduced pressure during curing of the microparticles in the laboratory reactor at 5° C. on encapsulation yield was tested. Three microparticle preparations were produced under varying conditions in accordance with Example 3 using the S/O/W-method. In preparation 1 the microparticles were cured at atmospheric pressure, in preparation 2 at 500 mbar, and in preparation 3 at 100 mbar. In all three preparations curing was carried out at 5° C. The effective active ingredient load of the microparticle preparations was determined according to the method described in Example 4 and from this the encapsulation yield (EY) was calculated. The results are shown in FIG. 1. Encapsulation yield increases with decreasing pressure.

### EXAMPLE 7

As in Example 6, microparticle preparations produced in a laboratory reactor under varying conditions were tested with respect to their encapsulation yield. In preparation 1 the microparticles were cured at atmospheric pressure, in preparation 2 at 500 mbar. In both preparations curing was carried out at 20° C. Encapsulation yield was then determined. As can be seen in FIG. 2, even at a processing temperature of 20° C. encapsulation yield increases with decreasing pressure.

### EXAMPLE 8

Microparticles were produced under three different conditions in a laboratory reactor in accordance with the S/O/W-method. In preparations 1 and 2 nitrogen was injected into the laboratory reactor during curing of the microparticles at 5° C. and 20° C. In preparation 3 the solvent was evaporated during the curing phase at 50° C. In vitro release of lysozyme in the microparticles of the three preparations was then determined in accordance with the method described in Example 5.

The results are shown in FIG. 3. When using higher temperatures a lower overall release is observable. By lowering the temperature from 20° C. to 5° C., initial release is reduced by 6% and overall release is increased to 99.7% after 1074 hours as opposed to 79.3% at 20° C.

### EXAMPLE 9

Five microparticle preparations were produced under varying conditions in accordance with the S/O/W-method:

20° C. during curing of the microparticles in a laboratory reactor under atmospheric pressure (“20° C.”)

5° C. during curing of the microparticles in a laboratory reactor under atmospheric pressure (“5° C.”)

20° C. during curing of the microparticles in a laboratory reactor at 100 mbar (“20° C. immediately at 100 mbar”)

5° C. during curing of the microparticles in a laboratory reactor at 100 mbar (“5° C. immediately at 100 mbar”)

in a beaker in accordance with Example 2, in which the outer phase was pre-cooled to 5° C., the S/O phase was dispersed in the outer phase and the S/O/W-emulsion was stirred at room temperature under atmospheric pressure. During the process the temperature of the curing microparticles adjusted to room temperature within 30 minutes (“5° C. with only initial pre-cooling in beaker”).

The in vitro release of lysozyme from the microparticles for the five preparations was then determined, the results of which are shown in FIG. 4.

Part of the results are summarized in the following Table 1:

TABLE 1

| | | | Linear released amount |
|---|---|---|---|
| | “Burst” after | Total release | (Difference between “burst” |
| | 5 h | after 912 h | and total release) |
| S/O/W beaker, | 27.5% | 100% | Approx. 72.5% |
| with initial pre- | | | |
| cooling at | | | |
| 5° C. | | | |
| Laboratory | 37.6% | 71.1% | Approx. 33.5% |
| reactor 20° C., | | | |
| 1013 mbar | | | |
| Laboratory | 26.1% | 85.5% | Approx 59.5% |
| reactor 5° C., | | | |
| 1013 mbar | | | |
| Laboratory | 17.6% | 62.8% | Approx. 45.2% |
| reactor 20° C., | | | |
| 100 mbar | | | |
| Laboratory | 22.4% | 90.5% | Approx. 68% |
| reactor 5° C., | | | |
| 100 mbar | | | |

In the beaker preparation a “burst” of 27.5% after 5 hours is observable. The “burst” at 20° C. and 1013 mbar is significantly higher at 37.6%. The “burst” is lower when the curing microparticles are cooled. Furthermore, a significantly higher total release of 85.5% is evident at 5° C. and 1013 mbar than at 20° C. and 1013 mbar following 912 hours of release. A vacuum can be applied to further reduce the release in the “burst”-phase.

### EXAMPLE 10

Two preparations of microparticles were produced independently of one another under identical conditions in a laboratory reactor according to the method described in Example 3. The conditions were: 5° C. and 100 mbar during curing of the microparticles.

The in vitro-release of both microparticle preparations was determined as in Example 5, the results of which are shown in FIG. 5. It is possible through reduplication to manufacture microparticles that have substantially the same release characteristics.

### EXAMPLE 11

Effect of pressure and temperature in conjunction with Leuprolin-MP under the W/O/W-method

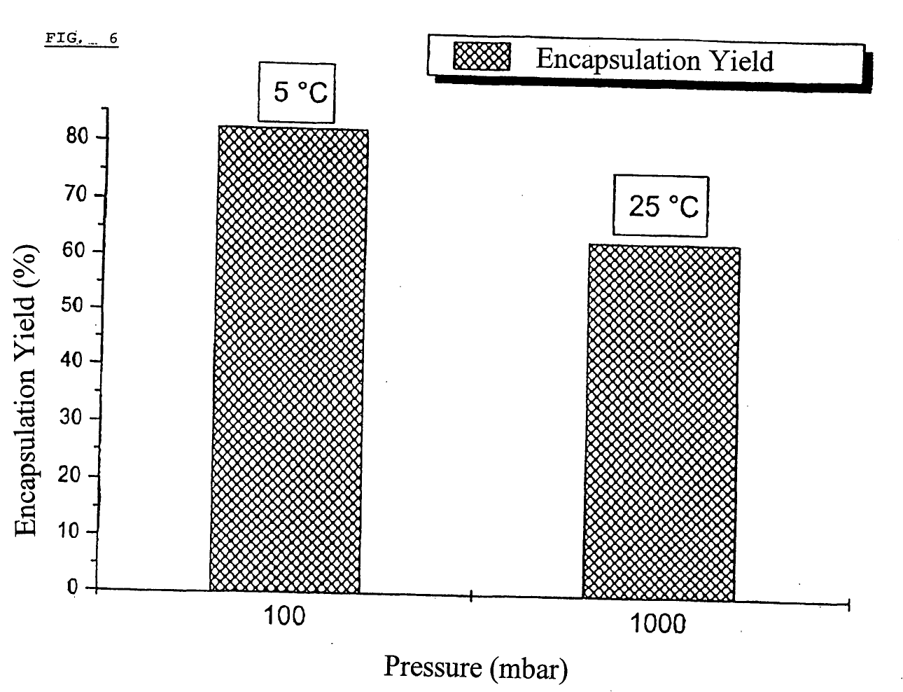

The effect of reduced pressure and temperature during curing of the microparticles in a laboratory reactor at 5° C. on microparticle characteristics was tested. As described in Example 1, two microparticle preparations were produced by the W/O/W method under varying conditions. The active ingredient used was leuprorelin acetate. In preparation 1 the microparticles were cured at 5° C. and 100 mbar, in preparation 2 at 25° C. and 1000 mbar. The effective ingredient load of the microparticle preparations was determined in accordance with the method described in greater detail in Example 4 and the resultant encapsulation yield (EY) calculated, the results of which are shown in FIG. 6. Encapsulation yield increases with decreasing pressure.

### EXAMPLE 12

Effect of Pressure, Temperature and Addition of Chitosan

The effect of reduced pressure and temperature during curing of the microparticles in a laboratory reactor at 5° C. on microparticle characteristics was tested. As described in Example 1, a microparticle preparation with the addition of chitosan (MW=150,000) was produced by the W/O/W-method. The active ingredient used was leuprorelin acetate.

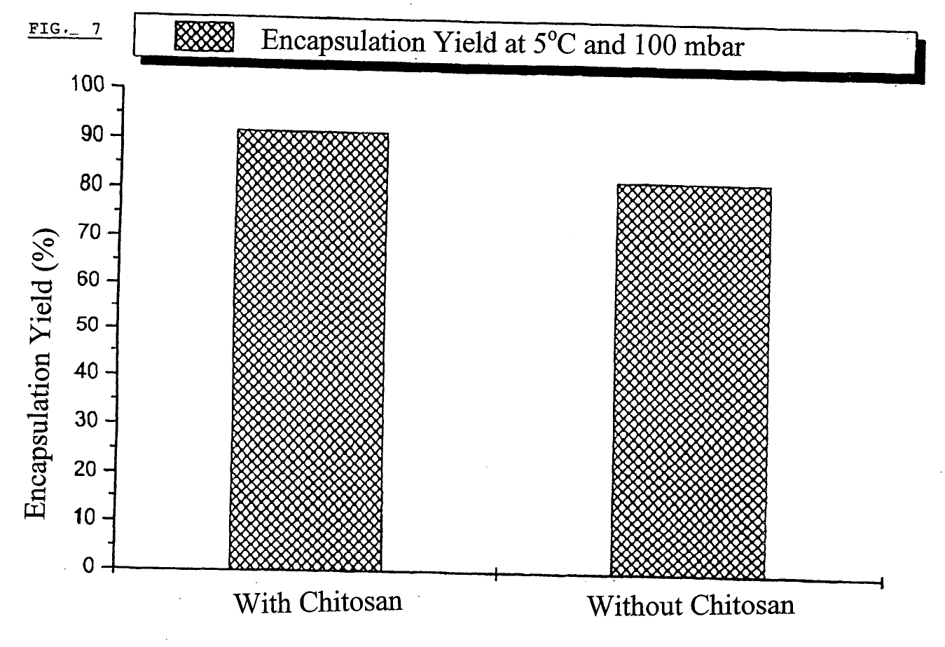

In preparation 1 the microparticles were cured at 5° C. and 100 mbar. The effective ingredient load of the microparticle preparations was determined as in the method described in Example 4 and the resultant encapsulation yield (EY) calculated, the results of which are shown in FIG. 7.

It is evident in this case that, unlike preparation 1, Example 11 (preparation by W/O/W without chitosan additive, but under temperature and vacuum) the results were elevated EY and a delayed release. This preparation shows that even better results may be obtained by the addition of chitosan.

### EXAMPLE 13

Effect of Pressure and Temperature in Conjunction with Leuprorelin Acetate Microparticles by the S/O/W-Method

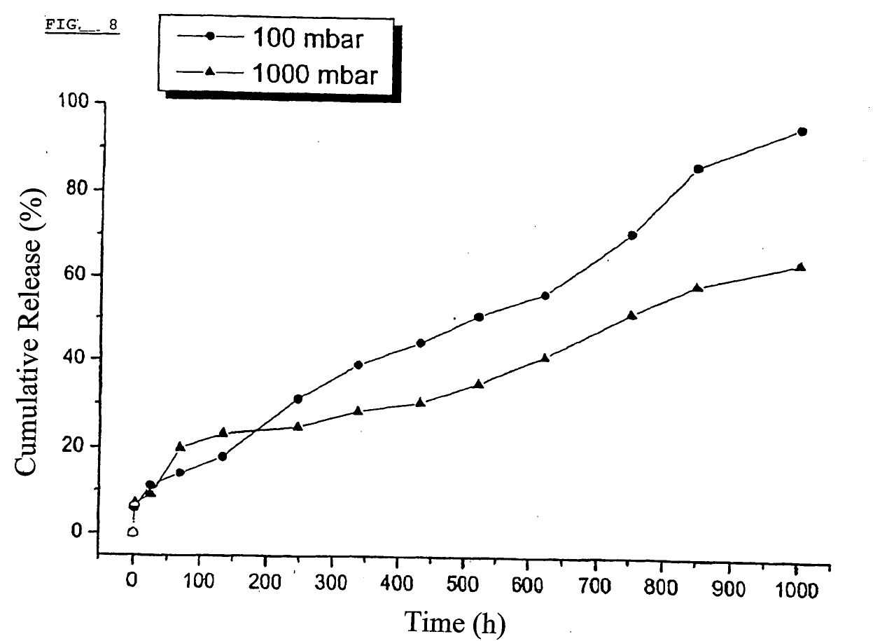

The effect of reduced pressure and temperature during curing of the microparticles in a laboratory reactor at 5° C. on microparticle characteristics was tested. Two microparticle preparations were produced by the W/O/W method described in Example 2 under varying conditions. The active ingredient used was leuprorelin acetate. In preparation 1 the microparticles were cured at 5° C. and 100 mbar, and in preparation 2 at 25° C. and 1000 mbar. The effective ingredient load of the microparticle preparations was determined as in the method described in Example 4 and the resultant encapsulation yield (EY) calculated. When applying a vacuum and low temperature the EY is higher by a factor of 2.25. The in vitro-release of the microparticles with Leuprorelin acetate is shown in FIG. 8.

What is claimed is:

1. Microparticles for the delayed release of an active ingredient, containing a polymer matrix and at least one physiologically active ingredient, characterized in that in accordance with the in vitro-release profile of said microparticles
a) less than 25% of the total amount of active ingredient is released within 24 hours of the onset of release; and
b) at least 80% of the total amount of active ingredient is released within 900 hours of the onset of release.

2. Microparticles according to claim 1, characterized in that in accordance with the in vitro-release profile of said microparticles, less than 20% of the total amount of active ingredient is released within 24 hours of the onset of release.

3. Microparticles according to claim 1 or 2, characterized in that in accordance with the in vitro-release profile of said microparticle, at least 90% of the total amount of active ingredient is released within 900 hours of the onset of release.

4. Microparticles according to one of the preceding claims, characterized in that release during the period between 24 hours and 900 hours of onset of release is kinetically substantially on the order of zero.

5. Microparticles according one of the preceding claims, characterized in that during the period between 48 and 900 hours of onset of release, 1.75% to 2.5% of the total amount of active ingredient is released daily.

6. Microparticles according to one of the preceding claims, characterized in that the polymer matrix consists essentially of polylactic acid, polyglycolic acid, a lactic acid-glycolic acid-copolymer or a mixture of at least two of the aforementioned components.

7. Microparticles according to one of the preceding claims, characterized in that contained therein is a physiologically active substance in the form of a peptide or protein.

8. Microparticles according to one of the preceding claims, characterized in that also contained therein is chitosan.

9. Method for manufacturing microparticles for delayed release of an active ingredient, characterized in that
a) a composition containing the active ingredient is added to an organic solution of a polymer and dispersed therein,
b) the emulsion or dispersion produced in a) is added to an outer phase and dispersed therein, whereby said the temperature of the outer phase at the time of addition is between 0° C. and 20° C., and
c) the organic solvent is removed by subjecting the dispersion or emulsion produced in b) to a pressure of less than 1,000 mbar, or by conducting an inert gas into the dispersion or emulsion produced in b).

10. Method according to claim 9, characterized in that the temperature is between 0° C. and 10° C.

11. Method according to claim 10, characterized in that the temperature is between 3° C. and 7° C.

12. Method according to one of the claims 9 to 11, characterized in that the dispersion or emulsion produced in b) continues to be regulated at a temperature of between 0° C. and 20° C. during removal of the organic solvent.

13. Method according to claim 12, characterized in that the dispersion or emulsion produced in b) continues to be regulated at a temperature of between 0° C. and 10° C. during removal of the organic solvent.

14. Method according to one of claims 9 to 13, characterized in that the organic solvent is removed by subjecting the dispersion or emulsion produced in b) to a pressure of 50 to 150 mbar.

15. Method according to one of claims 9 to 13, characterized in that the organic solvent is removed by conducting an inert gas, preferably nitrogen, into the dispersion or emulsion produced in b).

16. Method according to one of claims 9 to 15, characterized in that a polymer in the form of polylactic acid, polyglycolic acid or a lactic acid-glycolic acid-copolymer is used.

17. Method according to one of claims 9 to 16, characterized in that the organic solution of a polymer contains a solvent in the form of dichloromethane.

18. Method according to one of claims 9 to 17, characterized in that the polymer concentration in the organic solution of a polymer is 5 to 50% (w/v).

19. Method according to one of claims 9 to 18, characterized in that the composition containing the active ingredient is an aqueous solution.

20. Method according to one of claims 9 to 18, characterized in that the composition containing the active ingredient consists of solids.

21. Method according to claim 20, in which the composition containing the active ingredient is prepared by spray-drying a solution containing the active ingredient.

22. Method according to one of claims 9 to 21, characterized in that an aqueous solution is used as the outer phase.

23. Method according to claim 22, characterized in that the aqueous outer phase contains an emulsifier and/or a protective colloid.

24. Method according to claims 23, characterized in that the protective colloid is selected from the group consisting of polyvinyl alcohol, polyvinylpyrrolidone and polyethylene glycol.

25. Method according to one of claims 9 to 21, characterized in that said outer phase is a non-aqueous phase containing an emulsifier and/or a protective colloid.

26. Method according to claim 24, characterized in that said outer phase contains Span, Tween or Brij.

27. Method according to one of claims 9 to 26, characterized in that the composition containing the active ingredient also contains chitosan.

28. Microparticles obtained by a method according to one of claims 9 to 27.

29. Pharmaceutical containing microparticles according to one of the claims 1 to 8 or 28.

30. Pharmaceutical according to claim 29, characterized in that it is prepared for parenteral adminstration.
